(19) United States
(12) Patent Application Publication
(10) Pub. No.: US 2006/0234926 A1
(43) Pub. Date: Oct. 19, 2006

(54) Nucleic acid ligands and uses therefor

(76) Inventors: Peter Stockley, Leeds (GB); Christopher Adams, Leeds (GB); David Bunka, Leeds (GB); Roma Rambaran, Leeds (GB); Simon Phillips, Leeds (GB); Sheena Radford, Leeds (GB)

Correspondence Address: MYERS BIGEL SIBLEY & SAJOVEC, PO BOX 37428, RALEIGH, NC, 27627, US

(21) Appl. No.: 10/533,979

(22) PCT Filed: Nov. 5, 2003
(86) PCT No.: PCT/GB03/04798

§ 371 Date: May 5, 2006

(30) Foreign Application Priority Data
Nov. 6, 2002 (GB) 0225833.3

(51) Int. Cl. A61K 48/00; A61K 38/17; C07K 14/705; C07H 21/04; C12P 21/06

(52) U.S. Cl. 514/12; 514/44; 435/69.1; 435/320.1; 435/325; 530/350; 536/23.5

(57) ABSTRACT

The present invention relates to novel nucleic acid molecules or ligands or aptamers with affinities for specific target molecules, and uses of such molecules. The target molecules are fibrillar proteins in all forms of the protein that is to say its monomeric, pre-fibrillar, protofibrillar and mature fibrillar forms. The molecules of the present invention are therefore useful as diagnostic or therapeutic or screening agents, or as potential lead compounds for ralionalised drug design.

The present invention relates to novel nucleic acid molecules or ligands with affinities for specific target molecules, and uses of such molecules, especially but not particularly as diagnostic or therapeutic or screening agents, or as potential lead compounds for rationalised drug design.

### BACKGROUND TO THE INVENTION

A number of proteins undergo specific aggregation into amyloid fibrils in vivo leading to a variety of pathological disorders, collectively known as amyloidoses¹. These diseases are characterised by the deposition of normally soluble proteins or peptides into insoluble fibrillar arrays. About 20 different proteins have been identified to date as the fibrillar component in different human amyloid diseases. In some cases, the intact wild-type protein is involved (β2-microglobulin, (β2m), calcitonin, insulin and amylin), whilst in other cases (amyloid precursor protein (APP), lysozyme, transtyretin, gelsolin and others) mutated forms of the protein or protein fragments are involved. However, it is not known how normally soluble, proteins or peptides transform into the ordered array of β-strands typical of amyloid, although partial denaturation of the native protein or folding of more highly denatured states is thought to be a critical first step^{2,3}. Equally importantly, the specie(s) responsible for the toxicity of amyloid is/are also unknown. There is some evidence to suggest that early oligomers or other fibril assembly intermediates, rather than the fully assembled amyloid fibrils could be the toxic agent⁴⁻⁷.

In recent studies, it has been shown that amyloid-like fibrils can be generated in vitro from proteins not known to be associated with disease⁸, suggesting that this structure could be the ultimate ground state of all polypeptide chains. In accord with this view, the sequences of proteins and peptides known to be involved in human amyloid diseases also show no similarity in sequence or overall properties, for example secondary structure propensity and hydrophobicity. In all cases, the protein fibrils formed have a common overall architecture in which β-strands orient perpendicular to the fibre long axis into an array known as a cross-β structure. Fibrils are typically long, unbranched, approximately 10 nm in diameter and are formed from simpler units known as protofilaments or filaments. Amyloid fibrils thus give rise to a distinct X-ray fibre diffraction pattern, and are identified by their unique ability to bind the dyes Congo red and Thioflavin T (Thio-T), resulting in characteristic spectral changes. However, fibril formation by peptides and proteins requires specialised equipment, whilst the states of aggregation of pre-fibrillar forms of these species are difficult to detect by current methods. Therefore imaging of fibrillar species in tissue samples has hitherto been difficult and largely restricted to end stage species. There is therefore a need for simple, routine screening protocols of biological tissues for all aggregated and fibrillar species.

Examples of amyloidosis disease include new variant CJD, mature onset diabetes, Alzheimer's disease and dialysis-related amyloidosis (DRA). Alzheimer's disease is characterised by large plaques in the brain that contain necrotic neurons, neurofibrillary tangles containing the tau protein and fibrils composed of peptides derived from the Alzheiner's Precursor Protein (APP)⁴. With regard to the treatment of Alzheimer's disease, studies with monoclonal antibodies directed against Aβ1-40 have shown that neurodegeneration can be halted and/or reversed in animal models, suggesting that vaccination against Aβ1-40 could be a successful therapy⁹. Unfortunately despite the promising initial animal work, recent clinical trials of this therapy failed due to cross-reactivity with the APP precursor protein leading to inflammation of the brain. Accordingly there is still no effective therapy for this disease.

Dialysis-related amyloidosis (DRA) involves the human protein, β₂m, the aggregation of which into amyloid fibrils is the cause of the disorder dialysis-related amyloidosis ¹⁰. This protein forms the non-covalently bound light chain of the class I MHC complex. As part of its normal catabolic cycle, β₂m dissociates from the heavy chain, whereupon it is carried in the serum to the kidneys where it is degraded and excreted. As a consequence of renal failure therefore, the concentration of circulating β₂m increases, and, by a mechanism currently unknown, the full-length wild-type protein aggregates to form amyloid fibrils that typically accumulate in the musculo-skeletal system. DRA is a common and serious complication of long-term haemodialysis, currently affecting more than 750,000 people world-wide and serious morbidity develops in more than 90% of patients undergoing dialysis for a period of 10 or more years. Despite the identification of β₂m as the culprit protein in DRA more than 16 years ago¹¹, there are currently no therapies for the disease other than organ transplantation.

Accordingly, determining the structure of amyloid fibrils, together with discovery of agents able to discriminate between the differing forms of amyloid aggregates, and being able to regulate or alter their aggregation properties would offer an immediate advantage for the design of therapeutic and diagnostic agents against amyloidosis. In addition, specific agents directed against either soluble Alzheimer's Aβ1-40 peptide or β₂m, or their proto- or fibrillar forms, that could prevent amyloid formation and hence halt progression, and even reverse, neuro-degeneration due to plaque deposition or β₂m amyloid build up would be of immediate therapeutic value of benefit to the pharmaceutical industry and sufferers of the diseases. Furthermore, if the 20 amyloidosis diseases have a common underlying molecular mechanism therapeutic reagents created against one type of amyloid could be useful against many others.

Novel synthetic DNA/RNA ligands, known as aptamers, have been defined¹² as artificial nucleic acid ligands that can be generated against amino acids, drugs, proteins and other molecules. They are isolated from complex libraries of synthetic nucleic acids by an iterative process of adsorption, recovery and reamplification.

RNA aptamers are nucleic acid molecules with affinities for specific target molecules. They have been likened to nucleic acid antibodies because of their ligand binding properties. They may be considered as advantageous agents over antibodies for a variety of reasons. Specifically, they are soluble in a wide variety of solution conditions and concentrations and their binding specificities are largely undisturbed by reagents that have significant effects on antibody reagents, e.g. detergents and other mild denaturants, moreover they are relatively cheap to isolate and produce compared to antibodies. They may also readily be modified to generate species with improved properties whereas antibodies cannot always be adapted easily. Extensive studies show that nucleic acids are largely non-toxic and non-immunogenic and aptamers have already found clinical application¹³, whereas antibodies being proteins are strongly immunogenic and require extensive and expensive manipulation to be used in humans.

However, a serious disadvantage associated with RNA aptamers is that natural RNAs are unstable in biological fluids. It is known in the prior art how to improve stability by chemically modifying RNAs so as to block nuclease action at 5′ and 3′ ends, and throughout the length of the molecule. However, such chemical modification can ultimately detrimentally alter the binding properties of the RNA and hence render them ineffective.

It is known from the prior art how to isolate aptamers from degenerate sequence pools by repeated cycles of binding, sieving and amplification. Such methods are described in U.S. Pat. No. 5,475,096, U.S. Pat. No. 5,270,163 and EP0533 38 and typically are referred to as SELEX (Systematic Evolution of Ligands by EX-ponential Enrichment). The basic SELEX system has been modified for example by using Photo-SELEX where aptamers contain photo-reactive groups capable of binding and/or photo cross-linking to and/or photo-activating or inactivating a target molecule. Other modifications include Chimeric-SELEX, Blended-Selex, Counter-SELEX, Solution-SELEX, Chemi-SELEX, Tissue-SELEX and Transcription-free SELEX which describes a method for ligating random fragments of RNA bound to a DNA template to form the oligionucleotide library. However, these methods even though producing enriched ligand-binding nucleic acid products, still produce unstable products. In order to overcome the problem of stability it is known to create enantiomeric “spiegelmers” (WO 01/92566). The process involves initially creating a chemical mirror image of the target, then selecting aptamers to this mirror image and finally creating a chemical mirror image of the SELEX selected aptamer. By selecting natural RNAs, based on D-ribose sugar units, against the non-natural enantiomer of the eventual target molecule, for example a peptide made of D-amino acids, a spiegelmer directed against the natural L-amino acid target can be created. Once tight binding aptamers to this target are isolated and sequenced, the Laws of Molecular Symmetry mean that RNAs synthesised chemically based on L-ribose sugars will bind the natural target, that is to say the mirror image of the selection target. This process is conveniently referred to as reflection-selection or mirror selection and the L-ribose species produced are significantly more stable in biological environments, are less susceptible to normal enzymatic cleavage and are nuclease resistant.

### STATEMENT OF THE INVENTION

According to a first aspect of the invention there is provided a purified and isolated non-naturally occurring nucleic acid ligand to a fibrillar protein, wherein said ligand is an RNA ligand selected from the group comprising:
(i) the nucleic acids depicted in any one of SEQ ID NOS: 1-55 or 58-105;
(ii) having the corresponding DNA or RNA sequences of any one of SEQ ID NOS: 1-55 or 58-105, or the corresponding fully complementary sequences thereof or their L-ribose derivatives;
(iii) derivatives of the sequence depicted in any one of SEQ ID NOS: 1-55 or 58-105 having at least about 60%, 70%, 80% or 90%, sequence identity to any one of the nucleotide sequences, and which have a binding affinity to a fibrillar protein.

Accordingly the nucleic acids of the present invention may be RNAs or their L-ribose derivatives or may be the DNAs encoding the RNAs or their L-ribose derivatives.

Reference herein to fibrillar protein is intended to include all forms of the protein that is to say its monomeric, pre-fibrillar, protofibrillar and mature fibrillar forms.

Sequence identity is the similarity between two nucleic acid sequences, or two amino acid sequences, and is expressed in terms of the percentage similarity between the sequences. The higher the percentage, the more similar the two sequences are. Homologues or orthologues of the protein, and the corresponding cDNA or gene sequence, will possess a relatively high degree of sequence identity when aligned using standard methods. This homology will be more significant when the orthologous proteins or genes or cDNAs are derived from species that are more closely related (e.g., human and chimpanzee sequences), compared to species more distantly related (e.g. human and C. elegaus sequences).

Reference herein to an aptamer is intended to include nucleic acid molecules with binding affinities for specific target molecules, especially but not exclusively RNA nucleic acid molecules.

Preferably, the nucleic acid ligand or aptamer is substantially homologous to and has substantially the same ability to bind said fibrillar protein as a ligand selected from the group comprising the nucleic acids depicted in any one of SEQ ID NOS: 1-55 or 58-105.

Preferably, the nucleic acid ligand or aptamer has substantially the same structure and the same ability to bind said fibrillar protein as a ligand selected from the group comprising the nucleic acids depicted in any one of SEQ ID NOS: 1-55 or 58-105.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

Reference herein to binding affinity is intended to include binding affinities expressed as equilibrium dissociation constants, Kd, in the from sub-millimolar to picomolar.

In one embodiment of the invention, we have investigated aptamers selected against β2m, Aβ1-40 and Aβ1-42, as both their L- and D-amino acid molecules, and their fibrillar forms. The monomeric targets being conveniently referred to as T1, the proto- and more mature fibrils as T2 and T3, respectively for the Aβ targets. The equivalent β2m targets being designated monomeric, “curly” fibrils and mature “rod-like” fibrils. Our initial aim was to better understand the molecular basis of sequence-specific recognition of RNAs by proteins and peptides. In the course of this work, we have not only isolated tight binding RNA aptamers against the targets but surprisingly and unexpectedly, the aptamers discovered have properties that make them potentially useful entities. This is in contrast to the species reported previously that bind amyloid plaques¹⁴.

Accordingly, one aspect of the invention pertains to isolated nucleic acid molecules (e.g., RNAs) comprising a nucleotide sequence which has a binding affinity for a fibrillar protein or biologically active portions thereof, as well as nucleic acid fragments suitable as primers or hybridization probes for the detection or amplification of nucleic acid ligands for fibrillar proteins. In particularly preferred embodiments, the isolated nucleic acid molecule comprises one of the nucleotide sequences set forth in SEQ ID NO: 1-55 or 58-105 or a complement thereof of one of these nucleotide sequences. In other particularly preferred embodiments, the isolated nucleic acid molecule of the invention comprises a nucleotide sequence which hybridizes to or shows at least about 50%, preferably at least about 60%, more preferably at least about 70%, 80% or 90%, and even more preferably at least about 95%, 96%, 97%, 98%, 99% or more homologous to a nucleotide sequence as in SEQ ID NOS: 1-55 or 58-105, or a portion thereof.

Preferably, the aptamers of SEQ ID NOS: 1 to 16 have a preferential binding affinity to a D-amino acid Aβ1-40 monomeric target.

Preferably, the aptamers of SEQ ID NOS: 17 to 36 have a binding affinity to a D-amino acid β1-40 pre-fibrillar target.

Preferably, the aptamers of SEQ ED NOS: 37 to 55 have a binding affinity to a D amino acid Aβ1-40 protofibril target.

Preferably, the aptamers of SEQ ID NOS: 58 to 71 have a binding affinity to native β2-microglobulin protein target. Preferably, the aptamers of SEQ ID NOS: 72 to 90 have a binding affinity to a β2-microglobulin immature fibril protein target.

Preferably, the aptamers of SEQ ID NOS: 91 to 105 have a binding affinity to a β2-microglobulin mature fibrillar protein target.

Preferably, the aptamers of the present invention further include any one or more of the following features as herein recited such as a fluorescent label, an imaging label or a flanking region.

The core for the aptamer is the random RNA oligonucleotide sequence, which is flanked by a 5′ and 3′ constant sequence (SEQ ID NOS:56, 57, 106 and 107) that provide primer hybridisation sites for Klenow extension, cDNA synthesis, polymerase chain reaction amplification and T7 RNA polymerase tanscritption, all of which are involved in the SELEX protocol. It should be appreciated that the selection of the constant flanking region is important to ensure optimum efficacy of SELEX. The 3′ flanking region acts as the attachment site for MMLV reverse transcriptase primer that converts the RNA aptamers to DNA. The 5′ flanking sequence acts as the point of attachment for PCR primers which initiate amplification of the selected sequence.

According to a yet further aspect of the invention there is provided a vector comprising or encompassing at least one or more aptamer of the present invention.

This aspect of the invention pertains to vectors, preferably expression vectors, containing a nucleic acid having a binding affinity for a fibrillar protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmild” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

According to a yet further aspect of the invention there is provided a host cell including at least one aptamer or a vector comprising at least one aptamer of the present invention.

According to a yet further aspect of the invention there is provided an aptamer binding motif comprising a peptide sequence derived from human β2m that retains the ability of the whole protein to form amyloid fibrils. This sequence, in either the natural L-amino acid or un-natural D-amino acid sequence, is NH₂-FYLLYYTE-COOH (SEQ ID NO: 111) or NH₂-DWSFYLLYYTEFT-COOH (SEQ ID NO:112) or NH₂-DWSFYLLYYTEFTPTEKDEYA-COOH (SEQ ID NO:113), where the designations NH₂ and —COOH show the chemical connectivity of the peptides and the sequence in between represents the standard one-letter code for the amino acids¹⁵. Note, in each case the amino terminus of these peptides can by either free or acetylated, and likewise the carboxy terminus can be either free or in the form of an amide.

In the course of investigating the aptamers of the present invention we have identified two fibrillar forms of the protein, an intermediate, globular (immature) form and a mature fibrillar form. Accordingly this new form represents a target for identifying/developing aptamers directed thereto and may be of value in identifying father agents that can control/prevent/reverse fibril formation.

According to a yet further aspect of the invention there is provided aptamers directed against the binding motif as herein before described.

According to a yet further aspect of the invention there is provided an aptamer directed against the cross β-core structure.

There is a common secondary structure in amyloids in that whatever the fold of the soluble form they share a secondary structure core composed of a β-strand orientated perpendicular to the fibril long axis. Thus, it is envisaged that aptamers directed against this target would find use as therapeutic agents in a variety of diverse amyloid diseases.

Currently twenty human proteins are known that cause disease via mis-folding and amyloid formation. It is estimated that amyloid formation is implicated in 1:1000 deaths in the UK and could be a major cause of tissue and organ ageing. Despite the variety of amyloid forming proteins the amyloid structure itself has a common secondary structural element kmown as a cross-β-fold that appears independent of the protein/peptide sequence involved, although this does alter the detailed morphology of the mature fibrils observed. Work with antibodies has identified common epitopes (recognition features) shared by many amyloid structures. We therefore believe that there is/are a common apatope(s) (aptamer recognition features) which will allow anti-amyloid aptamers to act as a generic “magic bullet”. It is believed that the anti-amyloid aptamers of the present invention have the potential to cross-react with all forms of amyloid whatever its source, thus dramatically extending their utility in imaging/screening and therapeutics.

According to a yet further aspect of the invention, there is provided a pharmaceutical composition comprising at least one aptamer of the present invention.

In another embodiment of the invention the pharmaceutical composition may comprise a number of aptamers each with binding affinities for the same or different forms of a fibrillar protein.

Preferably, the pharmaceutical composition includes a suitable excipient, diluent or carrier.

According to a yet further aspect of the invention there is provided use of an aptamer of the present invention for the manufacture of a medicament for treating amyloid diseases.

It is envisaged that pharmaceuticals and medicaments utilising the aptamers of the present invention may be used to treat Alzheimer's and DRA disease conditions.

According to a yet further aspect of the invention there is provided a method of treating a patient suffering from Alzheimer's disease or a disease associated with amyloid formation comprising administering a therapeutically effective amount of an aptamer or pharmaceutical composition comprising an aptamer of the present invention.

There is evidence that βA1-40 and Aβ1-42 fibril formation is naturally prevented by other systems that recognise the peptide and clear it from the blood stream. These systems seem to work less well in Alzheimer's patients. If self-aggregation can be slowed and/or prevented and the growing fibrils remain soluble, it should be possible for these natural systems to keep up with peptide clearance.

Kidney dialysis patients suffer from induction of fibril formation in their β2m, leading to painful deposition in joints. It is envisaged that the aptamers and pharmaceutical compositions of the present invention will be of use in treating such conditions.

Preferably, the aptamer or pharmaceutical composition comprising an aptamer is administered directly to an amyloid site or it may be administered by an intra-venous, intra-muscular, intra-peritoneal route and preferably may be administered on more than one occasion. In addition, aptamers could be administered to the blood stream, the site of the raised level of soluble β2m, in order to stablise the soluble form of the protein.

It will be appreciated that the aptamers against the Aβ1-40, Aβ1-42 and β2m species may be modified with fluorescent labels by simple inclusion of, for example, fluorescein-labelled UTP in in vitro tanscription reactions. The fluorescence properties of these molecules are sensitive to their bound state and may be the basis of simple diagnostic screening and imaging reagents. For instance, the state of disease progression may be judged by staining/screening with differently labelled aptamers directed against monomer, pre-fibril or fibrillar species and therefore preferably the aptamers and pharmaceutical compositions comprising one or more aptamers will have utility not only in treating disease conditions and ameliorating symptoms but in assaying disease prevention and progression.

According to a yet further aspect of the invention there is use of the aptamers of the present invention as a diagnostic agent for detecting the presence and/or progression of an amyloid disease.

Aptamers of the present invention directed against the fibrillar forms of β2m and the reflection-selection aptamers against the Aβ1-40 species or Aβ1-42 species may preferably be suitably modified with at least one fluorescent label to allow diagnostic screening and/or at least one imaging reagent for these conditions. For instance, the state of disease progression may be monitored by staining/screening with differently labelled aptamers directed against individual monomer, pre-fibril or fibrillar species or a mixture of species over period of time.

According to a yet further aspect of the invention there is provided a method of monitoring the presence and/or progression of an amyloid disease comprising administering to a patient at least one aptamer or a pharmaceutical composition or aptamer product comprising an aptamer according to the present invention and imaging the presence of binding of said aptamer to an amyloid fibril and optionally repeating the process at a later date to assess presence or progression of a disease state.

According to one aspect of the invention there is provided a method for the isolation of nucleic acid ligands to a fibrillar protein target comprising:
(i) preparing a candidate mixture of nucleic acids;
(ii) contacting the candidate mixture of nucleic acids with a biotinylated immobilised fibrillar protein, wherein nucleic acids having an increased affinity to the fibrillar protein relative to the candidate mixture may be partitioned from the remainder of the candidate mixture;
(iii) partitioning the increased-affinity nucleic acids from the remainder of the candidate mixture;
(iv) amplifying the increased-affinity nucleic acids to yield a mixture of nucleic acids with relatively higher affinity and specificity for binding to the fibrillar protein, whereby a nucleic acid ligand of the fibrillar protein may be identified.

Preferably, the candidate mixture comprises single stranded nucleic acids and more preferably the single stranded nucleic acids comprise ribonucleic acids.

Preferably, the target comprises at least one biotin molecule. The β2m target protein requires biotinylation in order for it to attach to a surface, for example a streptavidin bead, so as to be immobilised however, biotinylation of native β32m may hinder fibril formation hence we have performed this step on preformed fibrils. Our results have shown surprisingly that a combination of pH shift and biotinylation on ice does not destroy fibrils and that SELEX is advantageously improved.

Preferably, the fibrillar target is isolated and immobilised. We have found surprisingly that, as exemplified by β2m, increasing the solution pH>5 does not lead to dissociation of the fibrils as expected from prevailing teachings¹⁶, provided that the sample is maintained on ice for up to 10 minutes. Accordingly, the modified method of the present invention allows for a biotinylated target to be immobilised by pH shift.

Preferably, the method further includes the step of modifying the nucleic acid ligands with a fluorescent label and/or an imaging reagent. A non-limiting example of a suitable fluorescent labels is fluorescein-labelled UTP, and non-liming example of imaging agents are uranyl acetate, and radioactive technetium and indium-labelled species, for both in vitro and in vivo applications.

Preferably, the method further includes the step of flanking said aptamer with at least one further nucleic acid sequence comprising the nucleic acid as set forth in SEQ ID NO:56 and optionally the aptamer is flanked by a further nucleic acid sequence as set forth in SEQ ID NO:107. It will be appreciated that other constant flanking regions may be used and the composition of the flanking regions is not intended to limit the scope of the invention.

The method of the present invention advantageously allows for rapid selection and characterisation and is accomplished in vitro without recourse to animal work. It will be appreciated that aptamers of the present invention will have commercial application in many areas currently making use of antibodies, for example and without limitation, as diagnostic and screening tools and as therapeutic agents in a variety of different disease conditions.

Preferably, the product may be further modified as hereinbefore described.

### BRIEF DESCRIPTION OF THE FIGURES

FIG. 1 represents a schematic diagram showing the growth of cross-β strand structure from soluble monomeric precursors to generate amyloid fibrils via a conformational rearrangement (top and middle lines). Also shown are models for how aptamer reagents might inhibit fibrillogenesis, either by stabilising the monomeric form (top line, right hand side) or by directly blocking fibril growth (lower line).

FIG. 2 shows that an anti-fibril β2m aptamer (SEQ D NO 74), labelled with fluorescein-UTP undergoes differential quenching and wavelength shifting of fluorescence emission in the presence of differing forms of the β2m target.

FIG. 3A shows surface plasmon resonance binding curves (sensorgrams) of the anti-β2m fibril aptamer (SEQ ID NO 74) binding to: a) an underivatised flow cell (red, the signal falls due to differences in refractive index of the sample); b) a flow cell derivatised with native β2m (magenta, no detectable binding under these conditions); c) a flow cell derivatised with β2m fibrils green, strong binding with slow dissociation) or d) a flow cell derivatised with mature β2m fibrils (blue, result as in (c)); FIG. 3B shows the equivalent experiments for a native aptamer (SEQ ID NO 61). Note, the anti-native aptamers were not counter-selected against fibrils.

FIG. 4A shows a time course of Aβ1-40 fibril formation followed by transmission electron microscopy (TEM) and FIG. 4B a the same time course in a continuous assay utilising the increasing Thio-T fluorescence upon fibril binding.

FIGS. 5A and B show nitrocelluose filter-binding curves for the binding of anti-Aβ1-40 T3 aptamers (SEQ ID NO:55 and 38 respectively) to their cognate target. 5B also shows the effects of adding aptamer SEQ ID NO:38 to a mixture of Aβ1-40 undergoing fibril formation (left panel). The upper TEM shows the formation of extended fibrils after 16 hours, whereas when the aptamer is added to an aliquot of the same sample after 10 mins of fibril formation further fibril formation is severely inhibited.

FIG. 6 shows reflection-selection apatmer (SEQ ID NO:38) binding to a natural enantiomer of its initial target and indicates the potential of this bio-stable aptamer to block fibril formation.

FIG. 7 shows mass spectrometry data for biotinylated β2m fibrils. The data suggest that under the conditions employed 1-3 biotin residues are introduced per β2m monomer.

FIG. 8 shows thio-T assays of the various points of the β2m biotin derivatisation protocol and indicates clearly that fibrils are still present after modification.

FIG. 9 shows the nature of the fibrillar β2m selection targets. The immature (short, curly) fibrils (left-hand side) and the mature fibrils (long, straight) on the right. The images (lower diagrams) are TEM and atomic force micrographs, respectively. The Thio-T binding potential of each fibrillar form is shown in the top panels.

FIG. 10 illustrates the effects of aptamers on β2m fibril formation in vitro. -β2m in the absence of aptamers; □-β2m in the presence of an anti-immature fibril (SEQ. ID NO 74); ▪-β2m in the presence of the anti-monomer 10^{th} round aptamer pool (NR10) and as a negative control, ◯-β2m in the presence of the naïve starting pool for the aptamers.

FIG. 11 shows the structure of human β2m and the location of E strand target peptides.

FIG. 12 shows the amino acid sequence of the peptides of β₂m.

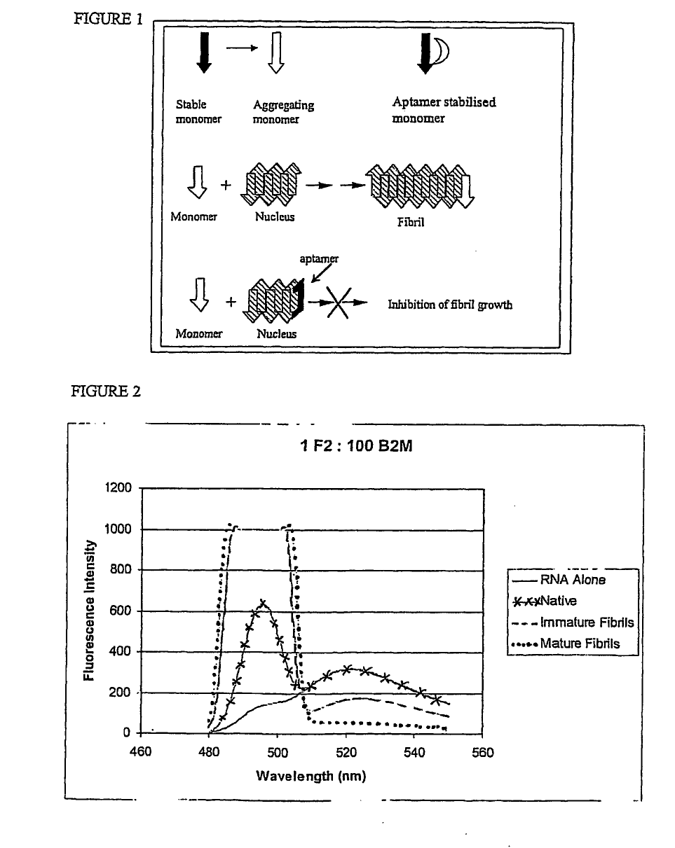

### MATERIALS AND METHODS

Aβ1-40 and Aβ1-42 Peptides

Fibril Formation

In order to characterise amyloid fibril formation from Aβ(1-40) using fluorescence assays and electron microscopy, thereby isolating and immobilising meta-stable targets for SELEX, the following method was employed. It will be appreciated that the method is also applicable for amyloid fibril formation from Aβ(1-42) target.

0.5 mgs of unbiotinylated Aβ(1-40) (obtained from Biosource) was dissolved in 1 mL 99.8% trifluoroacetic acid (TFA). The TFA was then removed by passing a jet of filtered, dry nitrogen gas over the sample. The dried Aβ was then dissolved in 1 mL hexafluoroisopropanol (HFIP, from Fluka) so as to remove any residual TFA. The HFIP was then similarly removed. The Aβ was then vacuum pump dried for 1 hr to remove any residual HFIP. The resulting disaggregated peptide was stored at −20° C. until required.

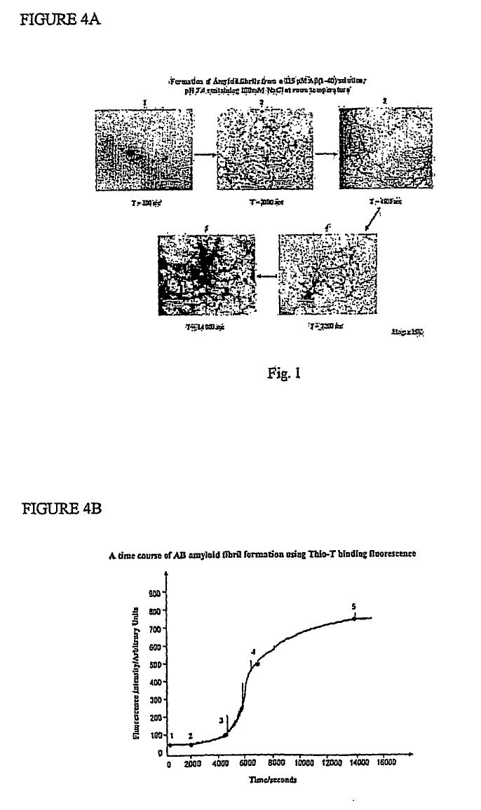

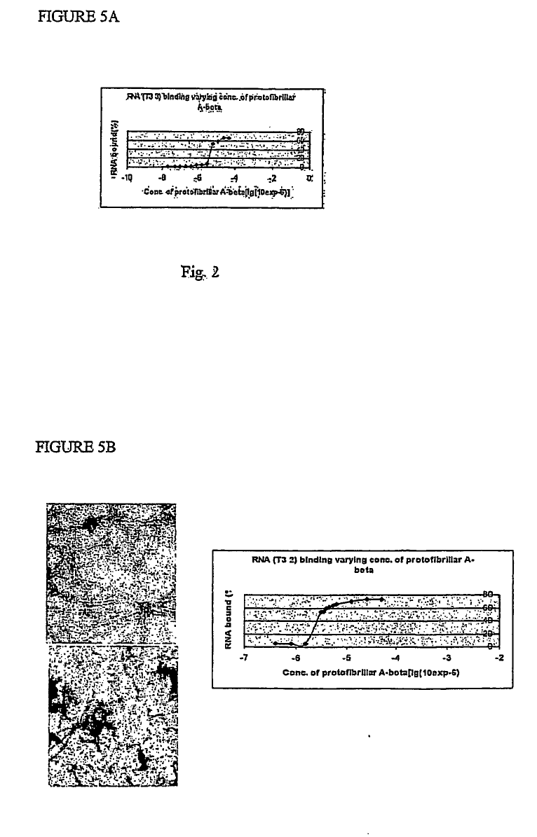

10:1 ‘mixed’ (unbiotinylated+biotinylated) Aβ fibrils were used from stocks of both forms of pre-prepared Aβ in HFIP (as described above). Appropriate volumes of each stock were mixed to produce a ˜50 μM (10:1) mixture of unbiotinylated and biotinylated Aβ in HFIP. The mixture was dried under N₂ gas and vacuum pump dried to remove all the HFIP. The ‘mixed’ treated peptide was then dissolved in an appropriate volume of cold 1× Binding Buffer (50 mM MOPS, pH 7.4, 50 mM NaCl, 1 mM MgCl₂) by rapid vortexing. The peptide solution was stored on ice and then quickly transferred to a 1 mL fluorescence cuvette containing a magnetic stirrer. A volume of Thio-T solution was added to a final concentration of 5 μM. The photobleaching shield from the fluorimeter (LS50B) was removed and a time drive scan initiated for 7200 seconds. The settings on the fluorimeter were λ_{EX}=444 nm λ_{EM}=482 nm S_{EX}=5 nm S_{EM}=10 nm. The scan was followed and 40 μL samples removed at t=100 s (corresponding to a steady fluorescence signal), 2000 s (corresponding to a slow increase in fluorescence signal), 4800 s (corresponding to an exponentially increasing in fluorescence signal); 7200 s (corresponding to a slowing of the rate of change of the fluorescence signal) and 14 400 s (corresponding to the approach of saturation by the fluorescence signal). Each of these samples were negatively stained and prepared for examination by electron microscopy (FIG. 4A).

The first meta-stable precursor species to fibril formation or 12 (appeared oligomeric under the EM) were identified by a steady fluorescence signal, lasting up to 2000 seconds. Subsequently, T2 was prepared by stirring the Aβ solution for 10 minutes and then storing on ice for immediate use in SELEX experiments.

The next meta-stable precursor species to be identified were the protofibrils or T3. These remained stable for up to 30 to 40 minutes after the formation of T1 (their presence was marked by a slow increase in fluorescence signal). The end of their existence is heralded by an exponential increase in fluorescence signal (marking the formation of elongating, maturing fibrils). T3 was prepared by stirring the Aβ solution for up to 40 minutes at 20° C. and then storing on ice for immediate use in SELEX experiments. T1 or monomeric Aβ(1-40) was stored in HFIP and subsequently diluted in 1× binding buffer when required.

With reference to FIG. 4A there is shown an time course of Aβ1-40 fibril formation followed by transmission electron microscopy (TEM), as will be apparent protofilaments are observed after 30-40 minutes of the procedure. FIG. 4B shows a corresponding time course of amyloid fibril formation using Thio-T binding fluorescence. Our results showed that both L- and D-amino acid versions of Aβ1-40 exhibited the same behaviour with respect to the kinetics and morphology of fibril formation

SELEX

In order to carry out at least 9 rounds of automated selection of RNA aptamers against three precursor meta-stable species of amyloid fibril formation T1 (native Aβ); T2 (oligomeric Aβ); T3 (protofibrillar Aβ), the following protocol was adopted. Using the following reagents, a T7 MEGA shortscript transcription kit [Ambion], RNase inhibitor [invitrogen], Upstream primer [5′ AAT TAA CCC TCA CTA AAG GGA ACT GTT GTG AGT CTC ATG TCG AA . . . 3′] (SEQ ID NO:56), Downstream primer [5′ TAA TAC GAC TCA CTA TAG GGA GAC AAG ACT AGA CGC TCA A . . . 3′] (SEQ ID NO:106) and Random 50 mer [5′ AAT TAA CCC TCA CTA AAG GGA ACT GTT GTG AGT CTC ATG TCG AA-N₅₀-TT GAG CGT CTA GTC TTG TCT 3′] (SEQ ID NO:107).

Each target was pre-prepared as hereinbefore described. Targets were immobilised onto Magnetic STREP-Microspheres as described by the manufacturer's instructions and stored at 4° C. until required. A program Amb T1-3 was written for the automated selection of RNA aptamers against all three targets. The program was modified from that of Cox and Ellington¹⁹ in the following manner:
(i) The DNA starting pool was prepared by amplifying a random 50 mer with the use of fixed upstream and downstream primer regions. The radioactively labelled pool was gel purified, quantified and subsequently used to generate the first RNA pool
(ii) Ambion kit transcription reagents (transcription buffer, NTPs, T7 polymerase) (Ambion) were used.
(iii) Mineral oil was used in the PCR step
(iv) Magnetic STREP-microspheres by Abgene were used instead of Dynabeads

Ligation and Cloning of RT-PCR Products Generated after 9 Rounds of Automated SELEX

RT-PCR products collected at the end of Round 9 of automated SELEX were amplified under the following PCR conditions: 10×PCR buffer, MgCl₂ (50 mM) to a final concentration of 1.5 mM; Taq DNA polymerase [Invitrogen] dNTP mix [Gibco BRL] to a final concentration of 0.02 mM; Upstream (SEQ ID NO:56) and Downstream primers (SEQ ID NO:106); Dimethyl Sulphoxide (DMSO); QIAEX II kit for gel purification [Qiagen]; TA TOPO Cloning system [(Invitrogen].

Fluorescently-Labelled Aptamers

A fluorescently-labelled T3 clone RNA (aptamer against target 3) (SEQ ID NO:38) was prepared and the effect on its fluorescence signal on binding to T3 was determined. The transcription mixture was prepared as follows: 100 ng DNA template 10 μLs; fluorescein RNA labelling mix [Roche] 2 μLs; 10× transcription buffer 2 μLs; RNase free water 18 μLs to 2 μLs; T7 RNA polymerase 2 μLs .

The mixture was incubated at 37° C. for 2 hrs. 2 μLs DNase I was then added to remove the template (incubation at 37° C. for 15 minutes). Reaction was stopped by adding 2 μLs 0.2 M EDTA, pH8. The transcript was purified by Phenol:Chloroform extraction and desalted by passing sample through a G-25 column (Amersham). The purified fluorescently-labelled RNA was EtOH precipitated to remove all unincorporated NTPs and stored in 10 μLs 1× Binding buffer at −20° C. until required.

A fluorescence emission scan (settings: λ_{EX}=495 nm, S_{EX}=3.0 nm S_{EM}=7.0 nm) was carried out on dilute labelled RNA (1:100 1× binding buffer), a control (1 pmol Fluorescein-12-UTP) was added and the change in fluorescence observed. Subsequently, T3 was added to labelled RNA in ratios of 1:2, 2:2, 4:2 and 6:2 and changes in fluorescence signals were observed.

Effect of T3 RNA Aptamer (SEQ ID NO:38) on the Fibril Formation Assay

In order to determine the effect of the T3 RNA aptamer (SEQ D NO: 38) on fibril growth assay, 600 μLs 50 μM fibril growth assay as hereinbefore described was prepared. The following reagents and apparatus were used: 10 mm×10 mm×4 mm Styrofoam (to provide increased height of cuvette); ˜440 μg purified unlabelled transcript of T3 clone (SEQ ID NO:38); dried, purified ‘Mixed’ Aβ(1-40); 1× binding buffer, magnetic stirrer; Fluorimeter LS 50B and; Formvar coated copper grids, 4% (w/v) uranyl acetate.

Fluorescence signal was observed and when the signal started to increase slowly after 30 minutes 20 μLs water was added and the effect observed. After a further 2-3 minutes the signal returned to baseline and 20 μLs T3.2 RNA (˜440 μgs) (SEQ ID NO: 38) was added and the effect observed. The experiment was repeated using ⅕^{th} amount T3 RNA (SEQ D NO: 38) (˜88 20 μs).

β2-Microglobulin Peptide

β₂ₘ Preparation & Fibril Formation

The β2m protein was over-expressed, extracted and purified as previously described¹⁶. Purification was carried out using an ÄKTA Explorer (Amersham Pharmacia Biotech) with a HiTrap Mono Q ion exchange column.

Immature fibril formation was carried out using a composite buffer consisting of 25 mM TRIS, 25 mM MES, 25 mM glycine, 25 mM sodium acetate (TMGA buffer). HCl was added to adjust to pH3.6 before the addition of sodium chloride to bring the final ionic strength to 400 mM. β₂M was added to a final concentration of 1 mg ml⁻¹ and the mixture incubated at 37° C. for 72 hrs. Unfolding and fibrillogenesis in β₂M can be triggered by altering pH and ionic strength.

Mature fibril formation was carried out using a composite buffer consisting of 25 mM sodium acetate & 25 mM sodium phosphate. HCl was used to adjust the pH to 2.5. No additional salt was included. β₂M was added to a final concentration of 2 mg ml⁻¹ and incubated at 37° C. on a shaker 150 rpm for 7 days.

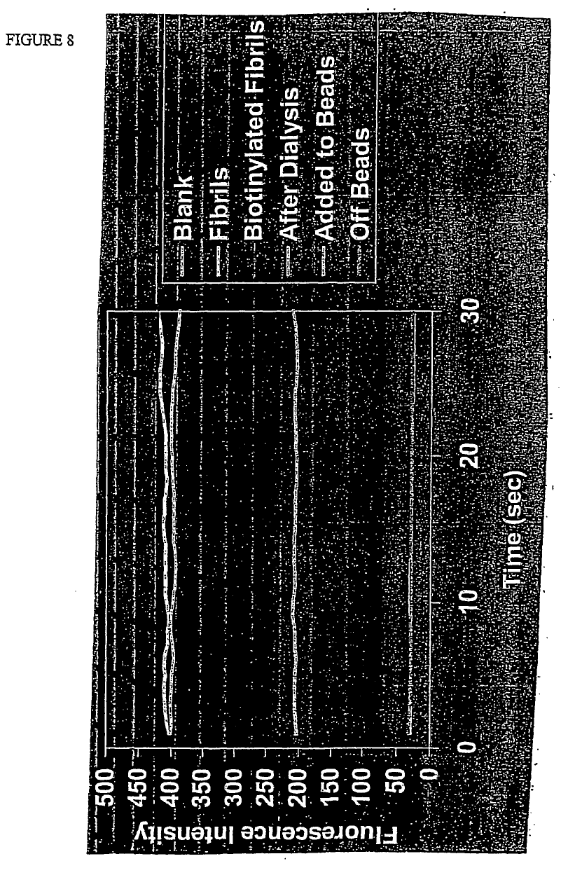

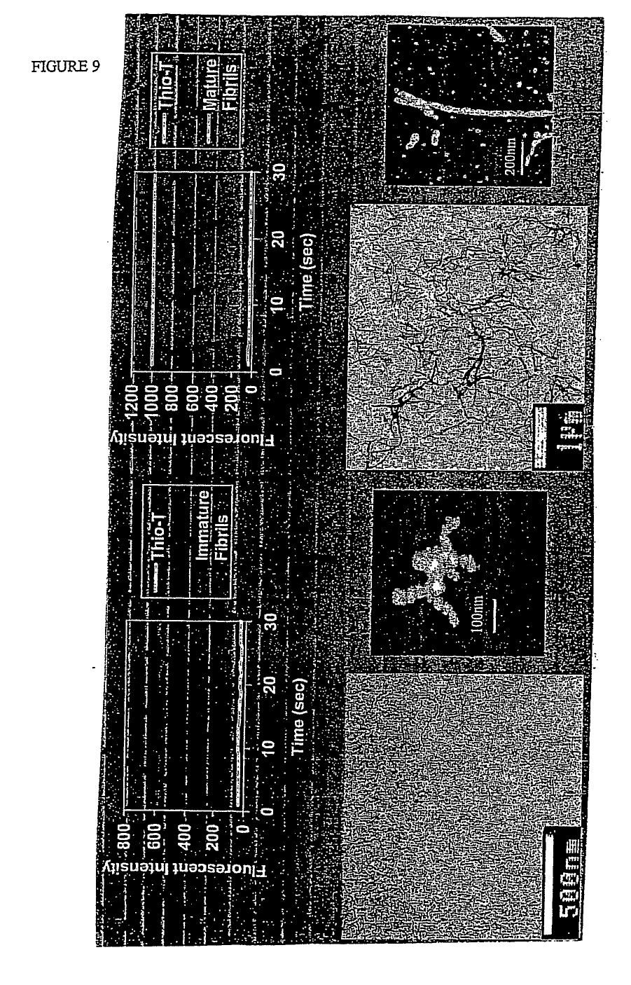

Fibrils were characterised by Thioflavin-T binding assay and electron microscopy. In both types of fibril formation reaction, a 10 μsample is removed and added to 990 μl 10 μM Thio-T buffered with 10 mM Tris HCl pH8.5. The average fluorescence emission signal over a 30 second scan is then measured using a Perkin-Elmer Luminescence Spectrometer (LS50 B) set at 444 nm (excitation) and 480 nm (emission). The presence of immature fibrils is indicated by a ˜10 fold increase in emission over that of Thio-T alone. Mature fibrils typically give a ˜20 fold increase over that of Thio-T alone. Addition of native state β₂M gives a marginal increase in fluorescence (FIG. 8),

Fibrils of both types can be viewed using electron microscopy. Copper-coated EM grids are placed coated side down onto a 30 μl drop of buffered fibrils for 30 seconds. Excess fibrils are removed by doting the grid onto a 30 μl drop of double distilled water, before placing the grid onto 4% (w/v) uranyl acetate for 45 seconds. Excess stain is removed by dotting onto a fresh drop of double distilled water. The grid is then allowed to air dry before viewing using a JEOL 1200 electron microscope.

Magnetic Bead Derivatisation

Native and fibrillar β₂M were biotin tagged using the ‘EZLink’ (Sulfo-NHS-LC-LC-biotin) reagent from PIERCE. The protocol supplied with the reagent had to be modified as the biotinylation reaction takes place at neutral pH (β₂M fibrils rapidly dissociate above pH5). ‘EZLink’ is dissolved in DMSO to a final concentration of 1 mg ml⁻¹. 30 μl of 2M Tris pH 10.8 is added to 500 μl of preformed fibrils (˜1 mg ml⁻¹) to raise the pH above 6. 75 μl of EZLink is added, mixed briefly by gentle pipetting and incubated on ice for 15 mins. The pH is reduced and excess NHS biotin is removed by placing the entire reaction into a ‘Slidalyzer’ cassette (3500 Mw cut off) and dialysing against 1 L fibril formation buffer for 2 hours. The dialysis buffer is changed after 1 hour. Streptavadin coated beads, purchased from Dynal, were derivatised with native β₂M, immature fibrils or mature fibrils as described in the Dynabeads protocol.

SELEX

All rounds of in vitro selection were carried out on a Biomek 2000 automated workstation (Beckman Coulter) using methods adapted from those described by Cox et al, ¹⁸. The Biomek has an integrated PTC-200 thermocycler with heated power bonnet (MJ Research), a multiscreen filtration system & vacuum manifold (Beckman Coulter) and a Thermal Exchange Unit (Beckman Coulter) with a Thermal 48 cooling block (Acme-Automation). The Biomek and all integrated components are controlled using Bioworks 3.1c (Beckman Coulter).

To produce the initial DNA pool; 20 μl 10×PCR buffer, 1.6 μl 10 mM dNTP's, 10 μl 10 μM sense primer JAN01P1 (P₁) (SEQ ID NO:108), 10 μl 10 μM anti-sense primer JAN01P2(P₂) (SEQ ID NO:109), 20 μl 0.1 μM template DNA JAN01T (SEQ ID NO:110), 6 μl 50 mM MgCl₂, 2 μl Taq DNA polymerase and 124.4 μl R⁻H₂O are thoroughly mixed and divided into 50 μl aliquots. These are then placed into a Techne Progene thermocycler and cycled as follow; 94° C. for 90 sec (Hot start), 8 cycles of: 94° C. for 45 sec (Denature); 50° C. for 60 sec (Anneal) and 72° C. for 90 sec (Extend)

As this model has a heated lid to prevent evaporation, the addition of mineral oil is not necessary The efficiency of PCR was assessed by polyacrylamide gel electrophoresis (PAGE).

RNA Pool

Transcription reactions were carried out by adding 5 μl 10× transcription buffer (to give final concentrations of 40 mM Tris-HCl pH7.9, 26 mM MgCl₂, 2.5 mM spermidine, 5 mM DTT, 0.01% Triton X-100), 16 μl R⁻H₂O, 16 μl 25 mM NTP mix, 10 μl template DNA. 40U RNasin and 100 U T7 RNA polymerase were then added to give a final volume of 50 μl. After mixing by rapid aspiration and dispensing, the transcription mix was incubated at 37° C. for 90 mins in the thermocycler.

Selection

20 μl derivatised Dynabeads (approximately 6.6×10⁶ beads) are added to 80 μl binding buffer and mixed by rapid aspiration and dispensing. The binding buffer used for the selections is the same TMGA buffer used for fibril formation. In the native β₂M selections, TMGA buffer pH7 is used. The entire transcription reaction is mixed into this. Mixing is repeated after a 5 min. room temperature incubation, to ensure the beads do not settle out. Partitioning of bound from unbound species is carried out by filtration through a multiscreen 96-well plate (PVDF membrane) supplied by Millipore. The Dynabeads are then rinsed by resuspending in binding buffer and repeating the filtration. Bound RNA species are eluted by resuspending the Dynabeads in 53 μl R⁻H₂O and incubating at 95° C. for 15 mins.

RT-PCR Amplification

The following are added to the eluted RNA; 5 μl 10 mM primer 1, 5 μl 10 mM primer 2, 2 μl 10 mM dNTPs and 33 μl RT-PCR buffer to give final concentrations of 10 mM Tris HCl pH8.4, 50 mM KCl, 5% acetamide, 0.05% Nonidet P40. This is heated to 65° C. for 10 mins. After reducing the temperature to 50° C., 200 U Superscript II™ reverse transcriptase and 5U Taq DNA polymerase are added. This is thermocycled as follows: 50° C. for 30 mins (Reverse transcribe), 8 cycles of: 94° C. for 45 see (Denature); 50° C. for 60 sec (Anneal) and72° C. for 90 sec (Extend).

10 μl of the RT-PCR product was used as a template in the transcription reaction for the next round. After 10 successful rounds of SELEX, samples of the RT-PCR products are cloned and sequenced.

Measuring Aptamer Binding Affinity by Surface Plasmon Resonance

Individual RNA aptamers are produced by PCR amplification and gel purification of the aptamer sequence from an individual clone. This is then used as the template DNA in a 50 μl transcription reaction (as carried out in vitro selections). Transcripts are DNase 1 treated to remove the template before phenol-chloroform extraction and ethanol precipitation.

All SPR experiments are carried out using a BIAcore 2000. TMGA buffer pH3.6 was used as running buffer. An SA chip (Dextran matrix pre-immobilised with streptavidin) was washed by carrying out three injections of 50 μl ‘chip preparation’ solution (50 mM NaOH, 1 M NaCl) at 50 μl min⁻¹ to remove any loosely bound streptavidin. Each flow-cell was derivatised with a different form of β₂M by injecting 150 μl ˜50 μM β₂M (Native, immature or mature fibrils) across a single flow-cell at 30 μl min⁻¹. Excess β₂M was removed by washing the flow-cells through with a 150 μl injection of running buffer.

### EXAMPLE 1

Aptamers N2 and F2 (SEQ ID NOS 61 & 74) were passed across each flow-cell (30 μl, 0.5 μM RNA at 10 μl min⁻¹). A selection of anti-native-β₂ₘ aptamers were passed across the blank and native β₂ₘ flow-cells at concentrations of 1 μM, 0.5 μM & 0.1 μM. The BIAevaluation software was used to correct the sensorgrams for Resonance Unit (RU) changes that are due to differences in the buffers rather than actual interactions. The software was then used to determine the Kd for each aptamer based on the corrected sensorgrams.

| Aptamer | Kd at 1 μM | | Kd at 0.5 μM | | Kd at 0.1 μM | |
|---|---|---|---|---|---|---|
| N2 (SEQ ID NO: 61) | 1.09 | μM | 1.91 | μM | 89.1 | nM |
| N4 (SEQ ID NO: 63). | | | 104 | nM | 317 | nM |
| N5 (SEQ ID NO: 64). | 785 | nM | 300 | nM | 845 | nM |
| N7 (SEQ ID NO: 66). | 2.58 | μM | 379 | nM | | |
| N8 (SEQ ID NO: 67). | 382 | nM | 343 | nM | 73.2 | nM |
| N11 (SEQ ID NO: 70). | 505 | nM | 329 | nM | 35 | μM |
| N13 (SEQ ID NO: 71). | 332 | nM | 1.46 | μM | 49.2 | nM |

### EXAMPLE 2

Our aptamers against the Aβ1-40 and β2m species (SEQ ID NOS: 1-55 and 58-71 and SEQ ID NOS: 72-90) have been modified with fluorescent labels by simple inclusion of fluorescein-labelled UTP in in vitro transcription reactions. With reference to FIG. 2 differential quenching and wavelength shifting in the presence of differing forms of the β2m target is shown with anti-fibril β2m aptamer (F2) (SEQ ID NO 74), labelled with fluorescein-UTP. The fluorescence properties of these molecules are sensitive to their bound state and may be the basis of simple diagnostic screening and imaging reagents. For instance, the state of disease progression may be judged by staining/screening with differently labelled aptamers directed against monomer, pre-fibril or fibrillar species.

### EXAMPLE 3

Anti-β2m aptamers have been isolated against native, monomeric protein as well as various amyloid fibrillar forms. Suitably modified versions of our aptamers against the non-fibrillar forms of β2m may be added to the blood during dialysis, stabilising that species with respect to fibril formation. Post-dialysis the aptamers will slowly hydrolyse in the body to harmless natural bi-products. Our anti-amyloid aptamers directed against β2m, were selected at each round after counter-selection against the native monomer. Remarkably, this approach has advantageously yielded reagents that bind to amyloid fibrils very tightly, dissociating very slowly, and in addition advantageously have little or no affinity for the native protein. Clearly, these reagents can be used directly to monitor early stages of amyloidosis in patients under-going dialysis. Since the anti-native aptamers do cross-react with the amyloid forms of the protein, these results show for the first time that apatopes of the native β2m are still present in the amyloid fibre. In addition, the fibril specific species appear to be recognising amyloid-specific apatopes, suggesting that these may be common to all amyloid fibrils, massively extending their utility.

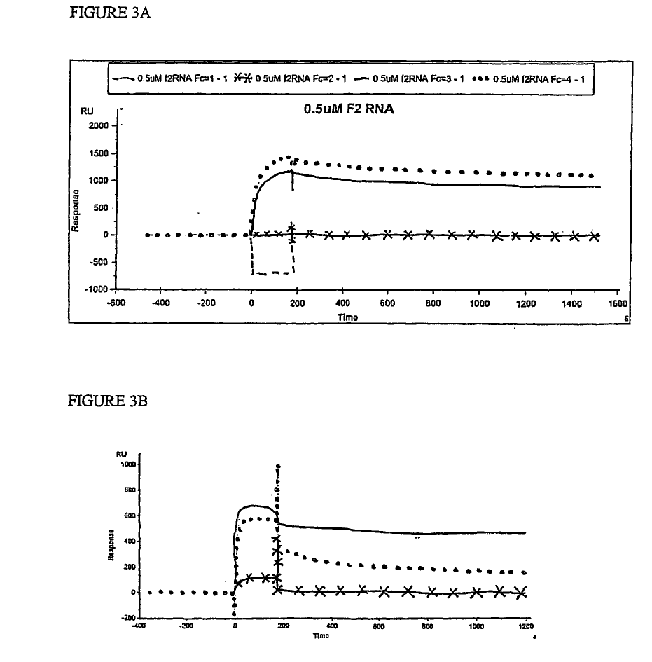

With reference to FIG. 3A we have shown by surface plasmon resonance binding curves (sensorgrams) of the anti-F2 β2m aptamer (SEQ ID NO 74) binding to: a) an underivatised flow cell (red, that the signal falls due to differences in refractive index of the sample); b) a flow cell derivatised with native β2m (magenta, that binding is detectable under these conditions); c) a flow cell derivatised with β2m fibrils (green, that there is strong binding with slow dissociation) and d) a flow cell derivatised with mature β2m fibrils (blue, result as in (c)).

With reference to FIG. 3 B, we have shown by a similar method the binding of an anti-native aptamer (SEQ ID NO 61) to the same immobilised targets using SPR. The sensorgram with native β2m as target (magenta) shows binding with rapid dissociation kinetics. This aptamer also binds strongly to the fibrillar targets suggesting that the apatopes being recognised is conserved after fibril formation. In the case of the fibrils the RU response is larger because there is more protein immobilised in these cases. Also, the dissociation behaviour is clearly distinct from that seen with the native monomer, being significantly slower. This is consistent with dissociation and rebinding because of the macromolecular nature of the fibrillar target.

### EXAMPLE 4

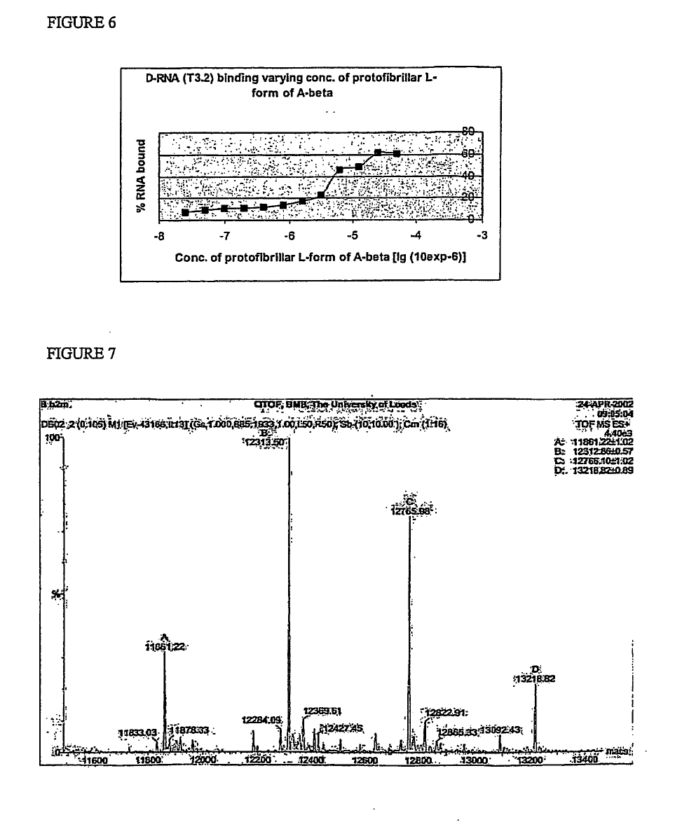

We have selected aptamer sequences against the D-amino acid forms of Aβ1-40. The self-aggregation behaviour of these non-natural Aβ1-40 peptides has been carefully assessed and three selection targets identified (FIG. 4). These are 1) T1 the monomeric peptide; 2) T2 the pre-fibrillar state and 3) T3 the protofibrils that are the direct precursors of mature filaments. This work therefore is a significant advance over the previous report of anti-Alzheimer plaques in which natural peptides were used and their state of aggregation was not assessed. Sequence comparisons of the aptamers isolated against each target suggest that there are amyloid specific apatopes in T2 and T3 that are not present in T1. The amyloid specific aptamers have some remarkable properties that make them potentially useful compounds. For example, one anti-T3 aptamer (SEQ ID NO: 38) binds its cognate target co-operatively (FIG. 5B), when labelled with fluorescence it shows specific fluorescence quenching upon amyloid binding, and therefore is a potential diagnostic and/or screening agent. It also dramatically reduces fibril formation when added to an in vitro fibrillation assay (FIG. 6).

### EXAMPLE 5

Experimental results with biotinylated β2m have shown that β₂m fibrils may be biotinylated with more than one biotin molecule. With reference to FIG. 7, peak A shows unbiotinylated β2m fibrils, the 452 Da gap to peak B is indicative of a single biotin molecule and the further 452 gap to peak C that the fibril carries a second biotin molecule. Similarly, the next 452 gap to peak D illustrates that the fibril can be associated with a third biotin molecule. Results have shown (FIGS. 8 and 9) that biotinylation of both immature and mature β2m fibrils allows the target to be successfully attached to a bead and that biotinylation does not affect the integrity or substantially alter the properties of fibrils compared to underivatised fibrils.

### EXAMPLE 6

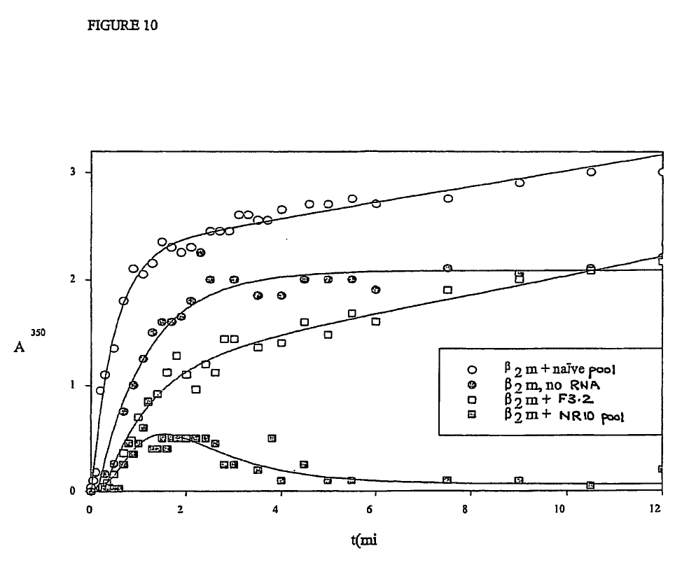

Studies on β2m fibril formation with two randomly selected aptamers F3.2 and NR10 (corresponding to SEQ ID NO 74 and the pool of aptamers generated after the 10^{th} selection round, respectively) have shown profound effects on fibril formation over time (FIG. 10). The anti-fibril aptamer, F3.2, shows distinct inhibitory properties with respect to fibril formation. Strikingly, the NR10 pool appears to allow only an initial burst of fibril formation followed by disassembly of the light-scattering species being generated, i.e. it appears capable of completely ablating fibril formation. Both aptamers were found to significantly reduce fibril formation compared to the control reaction of β2m with the naïve pool. Interestingly, β2m fibril formation appears to be enhanced in the control sample, which may indicate that there are species in the starting RNA pool that can actively promote fibril formation. This may provide a unique insight into the natural mechanism of amyloid formation in vivo.

### EXAMPLE 7

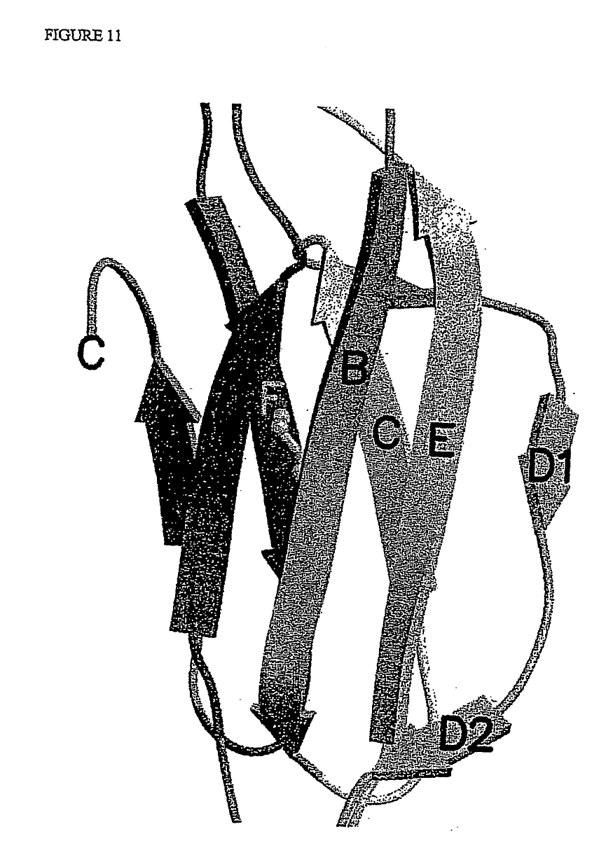

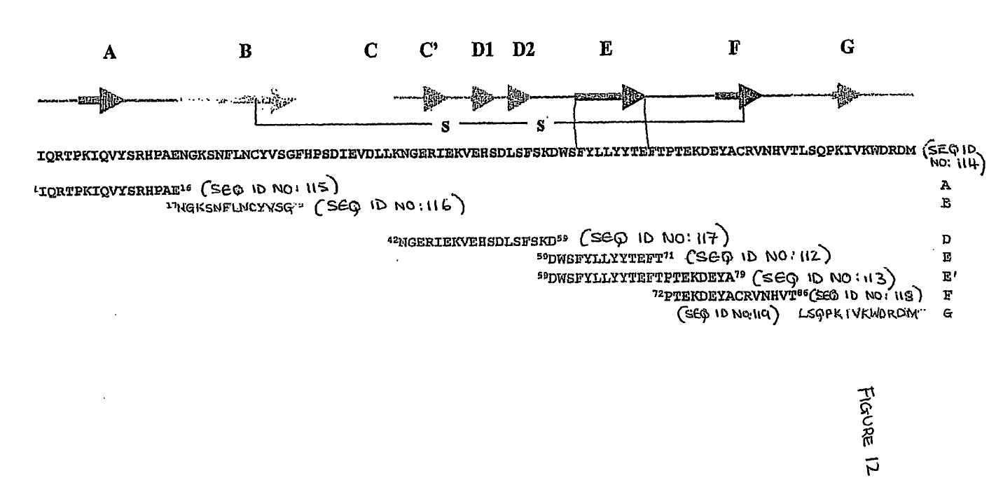

Structure of human β₂m is shown in FIG. 11 and corresponding the amino acid sequence is shown in FIG. 12, and is coloured from the N-terminus of the protein (red) to the C-terminus (violet). The location of elements of secondary structure was determined using DSSP. Individual-strands are labelled A though G. The disulphide bond that links Cys 25 (strand B) and Cys 80 (strand F) is also shown. The figure was made using the co-ordinates 1DUZ.PDB using the programme MOLSCRIPT and RASTER 3D. (b) The amino acid sequences of the peptides of β₂m studied here. With the exception of Ilel and Met99, all peptide sequences were acetylated at their N-termini and amidated at their C-termini.

Results have shown, only two sequences, both of which encompass the region that forms strand E in native β₂m, are capable of forming amyloid-like ffbrils in vitro. These peptides correspond to residues 59-71 (peptide E) and 59-79 (peptide E′) of intact₂m. The peptides form fibrils under the acidic conditions shown previously to promote amyloid formation from the intact protein (pH<3 at low ionic strength and pH<5 at high ionic strength), and also associate to form fibrils at neutral pH. Fibrils formed from these two peptides enhance fibrillogenesis of the intact protein.

Accordingly, we believe that residues 59-79 are important in the self-association of partially folded ₂m into amyloid fibrils and are potentially involved in the assembly mechanism of the intact protein in vitro. Thus, these residues represent a target against which aptamers may be directed and these residues provide a hitherto unrecognised target site for developing amyloid disease therapeutics.

### REFERENCES

1. Sunde, M. & Blake, C. C. (1998). From the globular to the fibrous state: protein structure and structural conversion in amyloid formation Q Rev Biophys 31, 1-39.
2. Fink, A. L. (1998). Protein aggregation: folding aggregates, inclusion bodies and amyloid Fold Des. 3, R9-R23.
3. Kelly, J. W. (1998). The alternative conformations of amyloidogenic proteins and their multi-step assembly pathways Curr. Opin. Struct. Biol. 8, 101-106.
4. Selkoe (1994) Normal and abnormal biology of the beta-amyloid precursor protein Ann. Rev. Neurosci., 17, 489-517;
5. Rochet et al. (2000). inhibition of fibrillization and accumulation of prefibrillar oligomers in mixtures of human and mouse alpha-synuclein Biochem. 39, 10619-10626.
6. Bucciantini et al. (2002). Inherent toxicity of aggregates implies a common mechanism for protein misfolding diseases Nature 416, 507-511.
7. Walsh et al. (2002). Amyloid-beta oligomers: their production, toxicity and therapeutic inhibition Biochem Soc Trans 30, 552-557.
8. Dobson, C. M. (1999). Protein misfolding, evolution and disease Trends in Biochemical Sciences 24, 329-332.
9. Check (2002) Nerve inflammation halts trial for Alzheimer's drug Nature, 415, 462.
10. Gejyo et al. (1986). Serum levels of beta 2-microglobulin as a new form of amyloid protein in patients undergoing long-term hemodialysis New Eng. J. Med 314, 585-586.
11. Gejyo et al. (1985). A new form of amyloid protein associated with chronic hemodialysis was identified as beta 2-microglobulin Biochem Biophys Res Commun 129, 701-706.
12. James W, Encyclopaedia of Analytical Chemistry. R. A Mayers (Ed) 4848-4871.
13. Rusconi et al. (2002) RNA aptamers as reversible antagonists of coagulation factor IXa Nature, 419, 90-94;
14. Ylera et al. (2002) Selection of RNA aptamers to the Alzheimer's disease amyloid peptide BBRC, 290, 1583-1588;
15. Jones et al. Amyloid-forming peptides from β2-microglobulin—Insights into the mechanism of fibril formation in vitro. J. Mol. Biol. in press.
16. Yamaguchi et al. (2001). Apolipoprotein E inhibits the depolymerization of β2-microglobulin-related amyloid fibrils at a neutral pH. Biochemistry, 40, 8499-8507.
17. McParland et al. (2000). Partially unfolded states of beta 2-microglobulin and amyloid formation in vitro Biochem. 280, 5678-5699.
18. Kad et al. (2001). Beta(2)-microglobulin and its deamidated variant, N17D form amyloid fibrils with a range of morphologies in vitro J Mol Biol 313, 559-571.
19. Cox et al (1998) Automated RNA selection Biotech. Prog., 14, 845-850

1. A purified and isolated non-naturally occurring nucleic acid ligand to a fibrillar protein target, wherein said ligand is an RNA ligand selected from the group consisting of:
(i) the nucleic acid depicted in any one of SEQ ID NOS: 1-55 or 58-105;
(ii) having the corresponding DNA or RNA sequences of any one of SEQ ID NOS: 1-55 or 58-105 or the corresponding fully complementary sequences thereof or their L-ribose derivatives; and
(iii) derivatives of the sequence depicted in any one of SEQ ID NOS: 1-55 or 58-105 having at least about 60%, 70%, 80% or 90% sequence identity to any one of the nucleotide sequences, and which have a binding affinity to a fibrillar protein.

2. The nucleic acid ligand according to claim 1 which is substantially homologous to and has substantially the same ability to bind said fibrillar protein target as a ligand comprising the nucleic acids depicted in any one of SEQ ID NOS: 1-55 or 58-105.

3. The nucleic acid ligand according to claim 1, wherein the nucleic acid has substantially the same structure and the same ability to bind said fibrillar protein target as a ligand selected from the group comprising the nucleic acids depicted in any one of SEQ ID NOS: 1-55 or 58-105.

4. The nucleic acid according to claim 1 wherein the fibrillar protein target is selected from the group consisting of monomeric β2m or Aβ1-40 or Aβ1-42, protofibrillar β2m or Aβ1-40 or Aβ1-42 and mature fibrillar β2m or Aβ1-40 or Aβ1-42.

5. The nucleic acid according to claim 1 wherein the fibrillar protein target comprises either L- or D-amino acid molecules.

6. The nucleic acid according to claim 1 wherein the nucleic acid of any one of SEQ ID NOS: 1 to 16 has a binding affinity to a D-amino acid Aβ1-40 monomeric target.

7. The nucleic acid according to claim 1 wherein the nucleic acid of any one of SEQ ID NOS: 17 to 36 has a binding affinity to a D-amino acid Aβ1-40 pre-fibrillar target.

8. The nucleic acid according to claim 1 wherein the nucleic acid of any one of SEQ ID NOS: 37 to 55 has a binding affinity to a D-amino acid Aβ1-40 protofibril target.

9. The nucleic acid according to claim 1 wherein the nucleic acid of any one SEQ ID NOS: 58 to 71 has a binding affinity to a native β2-microglobulin protein target.

10. The nucleic acid according to claim 1 wherein the nucleic acid of any one of SEQ ID NOS: 72 to 90 has a binding affinity to a β2-microglobulin immature fibril protein target.

11. The nucleic acid according claim 1 wherein the nucleic acid of any one of SEQ ID NOS: 91 to 105 has a binding affinity to a β2-microglobulin mature fibrillar protein target.

12. The nucleic acid according to claim 1 further comprising any one or more of the following features:
(i) a fluorescent label;
(ii) an imaging label; and
(iii) a flanking region.

13. The nucleic acid according to claim 12 wherein the flanking region comprises any one or more nucleic acid sequences selected from the group consisting of SEQ ID NOS: 56, 57, 106 and 107.

14. A vector comprising at least one or more nucleic acids of claim 1.

15. A host cell comprising at least one or more nucleic acids of claim 1 or a vector comprising the at least one or more nucleic acids of claim 1.

16. Use of a binding motif comprising a peptide sequence derived from human β2m that retains the ability of the whole protein to form amyloid fibrils, as a target for selecting a nucleic acid ligand.

17. Use of a peptide sequence comprising any one of SEQ ID NO: 111, 112 or 113 or derivatives or variants thereof that retain the ability of the whole protein to form amyloid fibrils, as a target for selecting a nucleic acid ligand.

18. A purified and isolated non-naturally occurring nucleic acid ligand to a fibrillar protein target, wherein the target comprises the binding motif of claim 16.

19. A purified and isolated non-naturally occurring nucleic acid ligand to a fibril cross β-core protein target.

20. A pharmaceutical composition comprising at least one nucleic acid of claim 1 or the vector comprising the at least one or more nucleic acids of claim 1.

21. A pharmaceutical composition according to claim 20 comprising at least two nucleic acid ligands each with binding affinities for the same or different forms of a fibrillar protein.

22. A pharmaceutical according to claim 20 further comprising a suitable excipient, diluent or carrier.

23. Use of a nucleic acid according to claim 1 for the manufacture of a medicament for treating amyloid diseases.

24. Use according to claim 23 for the treatment of Alzheimer's and DRA disease conditions.

25. A method of treating a patient suffering from Alzheimer's disease or a disease associated with amyloid formation comprising administering a therapeutically effective amount of (i) the nucleic acid ligand according to claim 1. (ii) a vector comprising the nucleic acids of claim 1, or (iii) a pharmaceutical composition comprising (i) or (ii).

26. A method according to claim 25 wherein the therapeutically effective amount of a nucleic acid ligand, vector or pharmaceutical composition is administered by an intra-venous, intra-muscular, intra-peritoneal route and optionally is administered on more than one occasion.

27. Use of the nucleic acid according to claim 1 or a vector comprising the nucleic acids of claim 1 as a diagnostic agent for detecting the presence and/or progression of an amyloid disease.

28. A method of monitoring the presence and/or progression of an amyloid disease comprising:
(a) administering to a patient (i) the nucleic acid ligand according to claim 1, (ii) a vector comprising the nucleic acids of claim 1, or (iii) a pharmaceutical composition comprising (i) or (ii);
(b) imaging the presence of binding of said nucleic acid ligand to an amyloid fibril; and
(c) optionally repeating the process at a later date to assess presence or progression of a disease state.

29. A method for the isolation of nucleic acid ligands to a fibrillar protein target comprising:
(i) preparing a candidate mixture of nucleic acids;
(ii) contacting the candidate mixture of nucleic acids with a biotinylated immobilised fibrillar protein on ice, wherein nucleic acids having an increased affinity to the fibrillar protein relative to the candidate mixture are partitioned from the remainder of the candidate mixture;
(iii) partitioning the increased-affinity nucleic acids from the remainder of the candidate mixture;
(iv) amplifying the increased-affinity nucleic acids to yield a mixture of nucleic acids with relatively high affinity and specificity for binding to the fibrillar protein, whereby a nucleic acid ligand of the fibrillar protein may be identified.

30. A method according to claim 29 wherein the candidate mixture comprises single stranded nucleic acids.

31. A method according to claim 30 wherein the single stranded nucleic acids comprise ribonucleic acids.

32. The method according to claim 29 further comprising modifying the nucleic acid ligand with a fluorescent label and/or an imaging reagent.

33. A method according to claim 29 wherein the nucleic acid ligand further comprises a flanking selected from the group consisting of SEQ ID NO: 56, 57, 107 and 108.

34. A nucleic acid product identified and isolated according to the method of claim 29.


SEQUENCE LISTING (113 sequences)

SEQ ID NO: 1 - DNA, 50 bp, Artificial Sequence
  synthetic ligand isolated from library
ggacgaagagcgttatcttacgctgactggatattagtttgttggaatac

SEQ ID NO: 2 - DNA, 43 bp, Artificial Sequence
  synthetic ligand isolated from library
tcgttattctgttcagggtttgagactcgttgtatcgatttag

SEQ ID NO: 3 - DNA, 41 bp, Artificial Sequence
  synthetic ligand isolated from library
ggcccctatacgaacgtcatcgtactcagctgttgttgtcc

SEQ ID NO: 4 - DNA, 47 bp, Artificial Sequence
  synthetic ligand isolated from library
tttatatcgcttccacgtgttttacccacaccttgttttagcggtaa

SEQ ID NO: 5 - DNA, 50 bp, Artificial Sequence
  synthetic ligand isolated from library
ttaatgacgctgtacatgggatcgtatagtgccacaggtaaatctgcatg

SEQ ID NO: 6 - DNA, 50 bp, Artificial Sequence
  synthetic ligand isolated from library
ctgttcttatcaagccaaataaagtactatgccctaaggcctatatgatc

SEQ ID NO: 7 - DNA, 50 bp, Artificial Sequence
  synthetic ligand isolated from library
cggtgggaaatatctttggctggcttagtaacttcgtatttgctcggctg

SEQ ID NO: 8 - DNA, 48 bp, Artificial Sequence
  synthetic ligand isolated from library
ttgtccattgtatacgatgcgcttcaaggatgatacggctttcacgca

SEQ ID NO: 9 - DNA, 50 bp, Artificial Sequence
  synthetic ligand isolated from library
tcccttagcaggcttcatttccttgaccattctttgcgttgagcctgtct

SEQ ID NO: 10 - DNA, 50 bp, Artificial Sequence
  synthetic ligand isolated from library
aaacgagtatctttttgggatctttcagctgctagtgcaatatcatctct

SEQ ID NO: 11 - DNA, 47 bp, Artificial Sequence
  synthetic ligand isolated from library
tggtcctgcgaccagcagtcaattagggtttagcacagtagaggcct

SEQ ID NO: 12 - DNA, 47 bp, Artificial Sequence
  synthetic ligand isolated from library
cgcgactaacttgcgtcagttttgtacgggtgtttagctccctttta

SEQ ID NO: 13 - DNA, 47 bp, Artificial Sequence
  synthetic ligand isolated from library
ttctctcacttccttccaggtgaacggcgcattaaggtcttgttgat

SEQ ID NO: 14 - DNA, 50 bp, Artificial Sequence
  synthetic ligand isolated from library
cagagttagatggcagctattgtcgcctaagtttgctttatatcatcttt

SEQ ID NO: 15 - DNA, 50 bp, Artificial Sequence
  synthetic ligand isolated from library
cggcgcgtccctgcaagtcaactgtttcctggctcgctttacgatccttt

SEQ ID NO: 16 - DNA, 50 bp, Artificial Sequence
  synthetic ligand isolated from library
cgtacacatcaaactttcgaatccacaggataatcgaggagatctcctga

SEQ ID NO: 17 - DNA, 50 bp, Artificial Sequence
  synthetic ligand isolated from library
ttgtcgcgatactacatttgtcctgtctgtgtgtcatacggaactgtgta

SEQ ID NO: 18 - DNA, 50 bp, Artificial Sequence
  synthetic ligand isolated from library
cgcccgtcgatttgaaacactcttcatcggcgttaaccacggcgtggaga

SEQ ID NO: 19 - DNA, 50 bp, Artificial Sequence
  synthetic ligand isolated from library
cgtaccgcgctacgttcccataagcgtgcaatttgtttgccttgggttgg

SEQ ID NO: 20 - DNA, 50 bp, Artificial Sequence
  synthetic ligand isolated from library
ctttcaaggtggggataccgagacaattagtcatctgtagtgtgatcagt

SEQ ID NO: 21 - DNA, 50 bp, Artificial Sequence
  synthetic ligand isolated from library
tagaagcgtcgaatcattgggaattcgtgcatttagggcaggcactttgg

SEQ ID NO: 22 - DNA, 49 bp, Artificial Sequence
  synthetic ligand isolated from library
caagcgaaatgtcactgtgtaaggtgtaacagggaacaagggaggacaa

SEQ ID NO: 23 - DNA, 50 bp, Artificial Sequence
  synthetic ligand isolated from library
ggtacttgttttcggtactttggtgctttgggaccacggtttgatggtgt

SEQ ID NO: 24 - DNA, 50 bp, Artificial Sequence
  synthetic ligand isolated from library
tgcgcggagtttctatttgtgtatggcgttccttatctagggcagacttc

SEQ ID NO: 25 - DNA, 50 bp, Artificial Sequence
  synthetic ligand isolated from library
tttgtacagtgctttcgcttgtcttcgcagcgcgctaattgcttgcgagt

SEQ ID NO: 26 - DNA, 50 bp, Artificial Sequence
  synthetic ligand isolated from library
gagctcagtggggtcttggtaggttagaagaagtagctatggctttcgaa

SEQ ID NO: 27 - DNA, 50 bp, Artificial Sequence
  synthetic ligand isolated from library
cgtgatagcccacggtcaagggtctacgagtcatttgttcctaacgctgc

SEQ ID NO: 28 - DNA, 50 bp, Artificial Sequence
  synthetic ligand isolated from library
caaggaggatatatgcttgggatcagtcgtgttgttgatatttatgatgg

SEQ ID NO: 29 - DNA, 50 bp, Artificial Sequence
  synthetic ligand isolated from library
aggctgcaggcttggatggtgcttcggggcgtcgtcttattgcctgctac

SEQ ID NO: 30 - DNA, 50 bp, Artificial Sequence
  synthetic ligand isolated from library
gcctcggtgtgttaaggaggaagagcgcccatctcagcagttaccggtcg

SEQ ID NO: 31 - DNA, 50 bp, Artificial Sequence
  synthetic ligand isolated from library
cgccccgtcgatttgaaacactcttcatcggcgttaaccacggcgtggag

SEQ ID NO: 32 - DNA, 50 bp, Artificial Sequence
  synthetic ligand isolated from library
cggactgagcattagttcgtgtcacagcgacgtcgttgtgtaaggctgaa

SEQ ID NO: 33 - DNA, 50 bp, Artificial Sequence
  synthetic ligand isolated from library
tcctgccaagctcctttttttggtagtgaatttcctttatcctggggcga

SEQ ID NO: 34 - DNA, 48 bp, Artificial Sequence
  synthetic ligand isolated from library
ctaaagtatgtgttagtatggccctgcttattaatgtcgggagttcgg

SEQ ID NO: 35 - DNA, 50 bp, Artificial Sequence; identical to SEQ ID NO: 21
  synthetic ligand isolated from library

SEQ ID NO: 36 - DNA, 50 bp, Artificial Sequence
  synthetic ligand isolated from library
taaagtgcggactcagggaataaggtgagaactgaccaaggtgtgtgttg

SEQ ID NO: 37 - DNA, 50 bp, Artificial Sequence
  synthetic ligand isolated from library
tcgacttttgacaagcatctcttttactgagagttgggagggaacggatg

SEQ ID NO: 38 - DNA, 50 bp, Artificial Sequence
  synthetic ligand isolated from library
ggggtgctaagcatttggcatccgttttgtgaactgctcgatgtttattc

SEQ ID NO: 39 - DNA, 50 bp, Artificial Sequence
  synthetic ligand isolated from library
ttaactgcaccttgcagtaactacaggatagcagtcctagagccagtcat

SEQ ID NO: 40 - DNA, 50 bp, Artificial Sequence
  synthetic ligand isolated from library
tgcattaccaggggataacctcgcatttggcgggttaggagtaacgtgca

SEQ ID NO: 41 - DNA, 50 bp, Artificial Sequence
  synthetic ligand isolated from library
actctcggtatattgttagattgggagcgacgcgtcaccgtatgacatgt

SEQ ID NO: 42 - DNA, 50 bp, Artificial Sequence
  synthetic ligand isolated from library
ggttagcggggttacctaacatcagctctagttagacctttttttacaac

SEQ ID NO: 43 - DNA, 50 bp, Artificial Sequence
  synthetic ligand isolated from library
cgaatgcttcgtagtaagtaatgaatctttttgacacactaatcgtagga

SEQ ID NO: 44 - DNA, 50 bp, Artificial Sequence
  synthetic ligand isolated from library
gtcccgtccatagtgctcgccaccatttcctttgggtcaggaattagttc

SEQ ID NO: 45 - DNA, 50 bp, Artificial Sequence
  synthetic ligand isolated from library
tgggagaaggactagctcgttattggtcatgttagtcatagcctgacacc

SEQ ID NO: 46 - DNA, 50 bp, Artificial Sequence
  synthetic ligand isolated from library
cggggcatagtacagaatcttagtgggtgttctttgattctggaggagca

SEQ ID NO: 47 - DNA, 50 bp, Artificial Sequence
  synthetic ligand isolated from library
gtgtgtttttatctactggtcggtggtttggttggtaatgcttaagcggc

SEQ ID NO: 48 - DNA, 50 bp, Artificial Sequence
  synthetic ligand isolated from library
ttggaacccagtgtgttttacacgctcatctttgcaacagggtgtgtgac

SEQ ID NO: 49 - DNA, 51 bp, Artificial Sequence
  synthetic ligand isolated from library
acggttaacaatcatgggcaagctcttagaacagcgttctgcgatttacga

SEQ ID NO: 50 - DNA, 50 bp, Artificial Sequence
  synthetic ligand isolated from library
gacgccgccggggagctctaaacggattaccatttgtcggtggtcttatt

SEQ ID NO: 51 - DNA, 50 bp, Artificial Sequence
  synthetic ligand isolated from library
gcataggatttcggttgggatagatattattcggtttcgatcatacgtat

SEQ ID NO: 52 - DNA, 50 bp, Artificial Sequence
  synthetic ligand isolated from library
ctcatagggatggtctcccgctgggctttttctgttaacatttagaactc

SEQ ID NO: 53 - DNA, 50 bp, Artificial Sequence
  synthetic ligand isolated from library
caggccgccggggagctctaaacggattaccatttgtcggtggtcttatt

SEQ ID NO: 54 - DNA, 51 bp, Artificial Sequence
  synthetic ligand isolated from library
tcacgtgtgctgtacgactgatgtttttgctgccagaatgactagaaatgt

SEQ ID NO: 55 - DNA, 50 bp, Artificial Sequence
  synthetic ligand isolated from library
atcgtaagtgattgttgttttacatggcctccagttggggagtcttccac

SEQ ID NO: 56 - DNA, 44 bp, Artificial Sequence
  synthetic ligand isolated from library
aattaaccctcactaaagggaactgttgtgagtctcatgtcgaa

SEQ ID NO: 57 - DNA, 39 bp, Artificial Sequence
  synthetic ligand isolated from library
ttgagcgtctagtcttgtctccctatagtgagtcgtatt

SEQ ID NO: 58 - DNA, 42 bp, Artificial Sequence
  Upstream primer against b2m
agtaatacgactcactatagggccaagcttgcatgcctgcag

SEQ ID NO: 59 - DNA, 20 bp, Artificial Sequence
  Downstram primer against b2m
ggtaccgagctcaggttccc

SEQ ID NO: 60 - DNA, 45 bp, Artificial Sequence
  synthetic ligand isolated from library
nattaggcgaacatagtagngaaagaaagnacagcangaggggct

SEQ ID NO: 61 - DNA, 51 bp, Artificial Sequence
  synthetic ligand isolated from library
gttggttacgcgtacagtgaaactcggaaagttgaagggccaaatgggaac

SEQ ID NO: 62 - DNA, 61 bp, Artificial Sequence
  synthetic ligand isolated from library
gagcagcaccgggccacggcgcaatcacctaaaatcggagccgccgggtgcagcatgtgac

SEQ ID NO: 63 - DNA, 61 bp, Artificial Sequence
  synthetic ligand isolated from library
ggcntgacanggcaggatagccggncnangcgancnntnggnccncganacccgacngtgg

SEQ ID NO: 64 - DNA, 42 bp, Artificial Sequence
  synthetic ligand isolated from library
ggtaccgaaaagtcattggttacaaccaggcaggcgcaggaa

SEQ ID NO: 65 - DNA, 45 bp, Artificial Sequence
  synthetic ligand isolated from library
gaatgggcgagttgagcgactgaggcagcatggaatcatacgtac

SEQ ID NO: 66 - DNA, 53 bp, Artificial Sequence
  synthetic ligand isolated from library
ggaggccgagagggcagaacccagcgacgcgacggaaagaccataacggcagc

SEQ ID NO: 67 - DNA, 61 bp, Artificial Sequence
  synthetic ligand isolated from library
gcacacgggccgcacccctgcgaaacgactatacgtcactagagcaggtttgcggagtccc

SEQ ID NO: 68 - DNA, 56 bp, Artificial Sequence
  synthetic ligand isolated from library
gtgcgagcagggcaaccagaagaaaacgtaggtagagtgtcgagttgatgagacgt

SEQ ID NO: 69 - DNA, 46 bp, Artificial Sequence
  synthetic ligand isolated from library
gccgcaagaacgggagaagagcggagaacgttgaaccgaccggagg

SEQ ID NO: 70 - DNA, 51 bp, Artificial Sequence
  synthetic ligand isolated from library
gtagagtgttgggctagcgtgaccagacatctacgtgggcagggagcattg

SEQ ID NO: 71 - DNA, 61 bp, Artificial Sequence
  synthetic ligand isolated from library
ggaggggccgtaacgcgaacgagacaaccagataaggaccgcccaatgagaggaacggcaa

SEQ ID NO: 72 - DNA, 122 bp, Artificial Sequence
  synthetic ligand isolated from library
agtaatacgactcactatagggccaagcttgcatgcctgcagatgagaatacacagaacagaggagaaggggatgaatcaataaggtcacactaaccaacncggtaccgagctcaggttccc

SEQ ID NO: 73 - DNA, 60 bp, Artificial Sequence
  synthetic ligand isolated from library
atgagaatacacagaacagaggagaaggggatgaatcaataaggtcacactaaccaacnc

SEQ ID NO: 74 - DNA, 60 bp, Artificial Sequence
  synthetic ligand isolated from library
aaaaataaantgtgtacacnaaanngcnanccccnggaangaangncaacaaangtnagt

SEQ ID NO: 75 - DNA, 59 bp, Artificial Sequence
  synthetic ligand isolated from library
gggagaattatggagtttgtaaaaccattggtcagtggcagaggttcatcaccgtcatc

SEQ ID NO: 76 - DNA, 60 bp, Artificial Sequence
  synthetic ligand isolated from library
acacctggaattaaattcaggacacccaacgcgcctacgaatagagcgggaacgacgacc

SEQ ID NO: 77 - DNA, 43 bp, Artificial Sequence
  synthetic ligand isolated from library
cggaagagcacaaaaaaggggactcgccttggaaggaacntgc

SEQ ID NO: 78 - DNA, 60 bp, Artificial Sequence
  synthetic ligand isolated from library
ttacgtcnggcctnaacgaatgaacggggaaagnaaggaatgattgggancatanacagn

SEQ ID NO: 79 - DNA, 15 bp, Artificial Sequence
  synthetic ligand isolated from library
ggagtgtggatatga

SEQ ID NO: 80 - DNA, 60 bp, Artificial Sequence
  synthetic ligand isolated from library
acacggaggcacctacagtactggggcaggagttgcgcagaggactagtggaaaaggagt

SEQ ID NO: 81 - DNA, 60 bp, Artificial Sequence
  synthetic ligand isolated from library
gccagcacgacaaanagcaggacgtaaagaagacgaaagggagaacattcggggagacga

SEQ ID NO: 82 - DNA, 60 bp, Artificial Sequence
  synthetic ligand isolated from library
cctgggatctccagggggcggcggagcgataggtgtgcgtgagttgaaaaggcagggcaa

SEQ ID NO: 83 - DNA, 60 bp, Artificial Sequence
  synthetic ligand isolated from library
gagacaagtcggtaggacagaggagtgaacaaaagaagcagagaggagagtcagagcagc

SEQ ID NO: 84 - DNA, 59 bp, Artificial Sequence
  synthetic ligand isolated from library
atctaccggttacaaatcaagagaactattttgtatgagccacttatctctcctgaagt

SEQ ID NO: 85 - DNA, 60 bp, Artificial Sequence
  synthetic ligand isolated from library
cgcangaaatataaacgtggggaggaccaggggagagtactccaaagagtgaagaaggtt

SEQ ID NO: 86 - DNA, 50 bp, Artificial Sequence
  synthetic ligand isolated from library
gcggtagaatgcatggcgattgcgattgtaggattccgcatcacacctcc

SEQ ID NO: 87 - DNA, 14 bp, Artificial Sequence
  synthetic ligand isolated from library
aaaaggacaggagc

SEQ ID NO: 88 - DNA, 59 bp, Artificial Sequence
  synthetic ligand isolated from library
actataagtnagnnggagggggancatgaaacaatngtttaagaacgtanantggntga

SEQ ID NO: 89 - DNA, 60 bp, Artificial Sequence; identical to SEQ ID NO: 80
  synthetic ligand isolated from library

SEQ ID NO: 90 - DNA, 53 bp, Artificial Sequence
  synthetic ligand isolated from library
aacgaaatgataacaacaaatacacacgtcacaccgaagttgacccaagactc

SEQ ID NO: 91 - DNA, 74 bp, Artificial Sequence
  synthetic ligand isolated from library
attaacgagantgaaggaaancgggagggagancaggcacaacggcgacangaaaagagagagtgcggtanccg

SEQ ID NO: 92 - DNA, 60 bp, Artificial Sequence
  synthetic ligand isolated from library
ccgaggccgcacacataagggggagggaaagacagcagaataaanaggaggcgatagtnc

SEQ ID NO: 93 - DNA, 57 bp, Artificial Sequence
  synthetic ligand isolated from library
ggtaaggtggaagggaaaaggaaacggaatgaacccgcgttatggccgaaaagggac

SEQ ID NO: 94 - DNA, 27 bp, Artificial Sequence
  synthetic ligand isolated from library
gaacaggacgcgtagccctacgatcca

SEQ ID NO: 95 - DNA, 57 bp, Artificial Sequence
  synthetic ligand isolated from library
cgaggacgaaanagtttagaaaaggagacggcgcttcgaacgaaccatcccctaggt

SEQ ID NO: 96 - DNA, 43 bp, Artificial Sequence
  synthetic ligand isolated from library
aagtgannaggcaaaaggaaaaaaaaggtgaacaaggctgctc

SEQ ID NO: 97 - DNA, 63 bp, Artificial Sequence
  synthetic ligand isolated from library
ggcccgacgaagtggggggaggaacgttngtgttgagtgattggagtnccctgggcgaaatcc

SEQ ID NO: 98 - DNA, 58 bp, Artificial Sequence
  synthetic ligand isolated from library
gcaaacgggggcgtttcggcggaggagtgggaagaaaaacgagagaagagttggaacg

SEQ ID NO: 99 - DNA, 37 bp, Artificial Sequence
  synthetic ligand isolated from library
gcanggggaangggattgaacgagaagaaggaggagc

SEQ ID NO: 100 - DNA, 64 bp, Artificial Sequence
  synthetic ligand isolated from library
gcacctttctcactttnngggcacaaaaccgcgcaccgggctagnctggagtgggatgaacgac

SEQ ID NO: 101 - DNA, 63 bp, Artificial Sequence
  synthetic ligand isolated from library
tgaggggaggttggcgcgaaggcgaggcagcacgaccagaagtttttgagttcgagaggggat

SEQ ID NO: 102 - DNA, 60 bp, Artificial Sequence
  synthetic ligand isolated from library
cctggaggagggcagcggncgagaaggggaaaaaagaaaagaactattcgtggagcggat

SEQ ID NO: 103 - DNA, 60 bp, Artificial Sequence
  synthetic ligand isolated from library
gaaaaggaggggatttnaaatcgcgtgaaaggtgaattgtttgccaggggcgaatncggt

SEQ ID NO: 104 - DNA, 66 bp, Artificial Sequence
  synthetic ligand isolated from library
ggggcttcatngagagaaggaaccacagacgaaaaaaagggggggngttcaagaggaagagaccga

SEQ ID NO: 105 - DNA, 60 bp, Artificial Sequence
  synthetic ligand isolated from library
gtgtaagcttgcgtcgaagatgaanaaaatgtcgaatcctcgctgtgccgccagacgtga

SEQ ID NO: 106 - DNA, 40 bp, Artificial Sequence
  Downstream primer
taatacgactcactatagggagacaagactagacgctcaa

SEQ ID NO: 107 - DNA, 64 bp, Artificial Sequence
  random 50 mer
aattaaccctcactaaagggaactgttgtgagtctcatgtcgaanttgagcgtctagtcttgtc

SEQ ID NO: 108 - DNA, 40 bp, Artificial Sequence
  sense primer
agtattacgactcactatagggccaagcttgcatgcctgc

SEQ ID NO: 109 - DNA, 20 bp, Artificial Sequence
  antisense primer
gggaacctgagctcggtacc

SEQ ID NO: 110 - DNA, 41 bp, Artificial Sequence
  template DNA
ccaagcttgcatgcctgcagnggtaccgagctcgaattccc

SEQ ID NO: 111 - protein, 8 aa, Homo sapiens
FYLLYYTE

SEQ ID NO: 112 - protein, 13 aa, Homo sapiens
DWSFYLLYYTEFT

SEQ ID NO: 113 - protein, 21 aa, Homo sapiens
DWSFYLLYYTEFTPTEKDEYA